(19) United States
(12) Patent Application Publication
(10) Pub. No.: US 2016/0270683 A1
(43) Pub. Date: Sep. 22, 2016

(54) SYSTEM FOR DETERMINING ELECTRICAL CHARACTERISTICS ON A SURFACE OF A HEART

(71) Applicant: KONINKLIJKE PHILIPS N.V., Eindhoven (NL)

(72) Inventors: MICHAEL GRASS, BUCHHOLZ IN DER NORDHEIDE (DE); FRANK MICHAEL WEBER, HAMBURG (DE); GERARDUS HENRICUS MARIA GIJSBERS, Liempde (NL); SANDER SLEGT, BEST (NL)

(21) Appl. No.: 15/032,332

(22) PCT Filed: Oct. 31, 2014
(86) PCT No.: PCT/EP2014/073408

§ 371 Date: Apr. 27, 2016

(30) Foreign Application Priority Data
Nov. 1, 2013 (EP) 13191239.6

(51) Int. Cl. A61B 5/042; A61B 5/0408; A61B 6/00; A61B 8/12; A61B 18/14; A61B 5/00; A61F 7/12; A61B 6/12; A61B 34/20; A61B 5/06; A61B 8/08

(52) U.S. Cl. CPC A61B 5/0421; A61B 5/065; A61B 5/04085; A61B 6/4441; A61B 8/12; A61B 8/0883; A61B 5/6853; A61B 5/061; A61F 7/12; A61B 5/0422; A61B 5/6823; A61B 6/12; A61B 8/0841; A61B 34/20; A61B 18/1492; A61B 2034/2051; A61B 2018/00023

(57) ABSTRACT

The invention relates to a system for determining electrical characteristics like electrical potentials on a surface of a heart (5). An esophageal electrode structure (6) measures electrical characteristics within an esophagus and a position determination unit (34) determines the position of the esophageal electrode structure within the esophagus and the position of the surface of the heart (5). The electrical characteristics on the surface of the heart (5) are then determined based on the measured electrical characteristics and based on the determined positions of the esophageal electrode structure (6) and the surface of the heart (5). Since for measuring the electrical characteristics the esophageal electrode structure (6) is used, the electrical characteristics can be measured within the esophagus and thus close to the surface of the heart (5), thereby allowing for an improved accuracy of determining the electrical characteristics on the surface of the heart (5).

### FIELD OF THE INVENTION

The invention relates to a system, method and computer program for determining electrical characteristics on a surface of a heart of a living being. The invention relates further to an interventional system for performing an interventional procedure within the heart of the living being.

### BACKGROUND OF THE INVENTION

For determining electrical potentials on a surface of a heart of a person electrocardiographic imaging may be used. A known electrocardiographic imaging method includes a) measuring electrical potentials on the anterior surface of the thorax of the person by using electrodes placed on the anterior surface, b) determining the positions of the electrodes and of the surface of the heart, for instance, based on a computed tomography image showing the electrodes and the surface of the heart, and c) calculating the electrical potentials on the surface of the heart based on the measured electrical potentials and the determined positions of the electrodes and the surface of the heart. This technique of electrocardiographic imaging has the advantage of providing electrical potentials on the surface of the heart, without requiring a measurement of these electrical potentials directly on the surface of the heart. However, the calculation of the electrical potentials on the surface of the heart is generally not very accurate.

### SUMMARY OF THE INVENTION

It is an object of the present invention to provide a system, method and computer program for determining electrical characteristics on a surface of a heart of a living being, which allow for a more accurate determination of the electrical characteristics on the surface of the heart. It is a further object of the present invention to provide an interventional system for performing an interventional procedure within the heart of the living being, which comprises the system for determining the electrical characteristics on the surface of the heart.

In a first aspect of the present invention a system for determining electrical characteristics on a surface of a heart of a living being is presented, wherein the system comprises:

an esophageal electrode structure being adapted to be introduced into the esophagus of the living being and to measure electrical characteristics within the esophagus,

a position determination unit for determining the position of the esophageal electrode structure within the esophagus and the position of the surface of the heart,

an electrical characteristics determination unit for determining electrical characteristics on the surface of the heart based on the electrical characteristics measured by the esophageal electrode structure and based on the determined positions of the esophageal electrode structure and the surface of the heart.

The accuracy of the determination of the electrical characteristics on the surface of the heart decreases with increasing distance between the electrodes measuring the electrical characteristics and the surface of the heart. Since for measuring the electrical characteristics the esophageal electrode structure is used, the electrical characteristics can be measured within the esophagus and thus close to the surface of the heart, thereby allowing for an improved accuracy of the determination of the electrical characteristics on the surface of the heart.

The system is preferentially adapted to perform electrocardiographic imaging. The measured electrical characteristics are preferentially electrical potentials. Thus, the esophageal electrode structure is preferentially adapted to measure electrical potentials within the esophagus, especially on a wall of the esophagus, wherein the measured electrical potentials are used by the electrical characteristics determination unit for determining electrical characteristics on the surface of the heart of the living being. The determined electrical characteristics on the surface of the heart of the living being are preferentially electrical potentials, particularly extracellular potentials, and/or locations of electrical activation wave fronts.

The system may further comprise an imaging unit for generating image data showing the esophageal electrode structure and/or the surface of the heart, wherein the position determination unit may be adapted to determine the position of the esophageal electrode structure and/or of the surface of the heart based on the generated image data. The surface of the heart, on which the electrical characteristics are determined, is preferentially not the surface of the entire heart, but the surface of an atrium, especially of the left atrium only. It may therefore not be necessary to image the complete heart and fewer electrodes may be used. The position determination unit is preferentially adapted to segment the surface of the heart and/or the esophageal electrode structure in the generated image data for determining the positions of the surface of the heart and/or of the esophageal electrode structure. Since in an embodiment both, the surface of the heart and the esophageal electrode structure, are segmented in the image data, the positions of the surface of the heart and the esophageal electrode structure can be reliably determined without necessarily requiring further tracking techniques for tracking the position of, for instance, the esophageal electrode structure. However, the position determination unit can also be adapted to use additional tracking techniques for determining the position of the esophageal electrode structure like optical shape sensing or electromagnetic tracking, wherein in this case the image may not show the esophageal electrode structure.

In an embodiment the system further comprises an outer electrode structure being adapted to be arranged on an outer surface of the living being, wherein the position determination unit is adapted to determine also the position of the outer electrode structure on the outer surface of the living being and wherein the electrical characteristics determination unit is adapted to determine the electrical characteristics on the surface of the heart based on the electrical characteristics measured by the esophageal electrode structure, the electrical characteristics measured by the outer electrode structure and the determined positions. The outer electrode structure is preferentially adapted for being arranged on the anterior surface of the thorax of the living being. It preferentially comprises several electrodes and a carrying element for carrying the electrodes. The outer electrode structure may be regarded as being a patch comprising the several electrodes. By considering also the electrical characteristics measured by the outer electrode structure while determining the electrical characteristics on the surface of the heart, the accuracy of the determination of the electrical characteristics on the surface of the heart can be further improved.

In an embodiment the system further comprises an imaging unit for generating image data showing the outer electrode structure on the outer surface of the living being, wherein the position determination unit is adapted to determine the position of the outer electrode structure on the outer surface of the living being based on the generated image data. In particular, the surface of the heart, the outer electrode structure and the esophageal electrode structure may all be segmented in the same image data for determining their positions. This allows for a relatively simple way of determining reliable positions. However, the position determination unit can also be adapted to determine the position of the outer electrode structure in another way, for instance, by using optical shape sensing tracking, electromagnetic tracking or another tracking technology, wherein in this case the image data may not show the outer electrode structure.

The imaging unit may be a C-arm x-ray unit for generating a three-dimensional image as the image data. The C-arm x-ray unit is preferentially adapted to perform a rotational angiography acquisition for acquiring x-ray projection data and to reconstruct the image based on the acquired x-ray projection data. The rotational angiography acquisition and the following reconstruction may be a three-dimensional atriography (3D ATG) procedure.

A contrast agent may be present in an atrium, particularly in the left atrium, of the heart and a three-dimensional image showing the distribution of the contrast agent within the atrium may be generated. This image may be used to segment the surface of the atrium and to also segment the electrodes of the esophageal electrode structure. This image may also be used to segment electrodes of the outer electrode structure. Based on these segmentations the positions can be determined, which can be used together with the measured electrical characteristics by the electrical characteristics determination unit for determining the electrical characteristics on the surface of the atrium. Since the electrodes and the surface of the heart are very well detectable in the reconstructed three-dimensional image, which has been reconstructed based on the acquired x-ray projection data, the positions of the electrodes and the surface of the heart can be determined very accurately, thereby allowing for a further improved accuracy of determining the electrical characteristics on the surface of the heart.

In an embodiment the imaging unit may alternatively or additionally comprise a transesophageal echocardiogram (TEE) ultrasound probe adapted to be introduced into the esophagus of the living being and to generate an ultrasound image as the image data. The TEE ultrasound probe may be a micro TEE ultrasound probe. Its width and height may be smaller than 10 mm, further preferred smaller than 5 mm and even further preferred smaller than 3 mm. The TEE ultrasound probe and the esophageal electrode structure may be separate components. However, in an embodiment they may be integrated. For instance, they can be integrated in a same esophageal catheter.

If the TEE ultrasound probe is used for imaging the surface of the heart of the person, a further imaging modality may not be necessarily required for determining the positions of the electrodes and the surface of the heart. In particular, using the TEE ultrasound probe can allow for determining the electrical characteristics on the surface of the heart, without necessarily requiring x-rays for imaging the electrodes and the surface of the heart, i.e. the radiation dose applied to the living being may be reduced. The position determination unit may be adapted to determine the position of the surface of the heart based on the image data showing the surface of the heart and in an embodiment the TEE ultrasound probe may be integrated in the esophageal electrode structure such that the spatial relation between the TEE ultrasound probe and the esophageal electrode structure is known, wherein the position determination unit may be adapted to determine the position of the esophageal electrode structure relative to the image data generated by the TEE ultrasound probe based on the known spatial relation between the TEE ultrasound probe and the esophageal electrode structure.

The esophageal electrode structure preferentially comprises electrodes and an esophageal carrying structure being adapted to carry the electrodes and to be introduced into the esophagus of the living being. The esophageal carrying structure is preferentially integrated with an esophageal catheter to be introduced into the esophagus. Moreover, the esophageal carrying structure is preferentially adapted such that the electrodes can be in contact with the wall of the esophagus, when measuring the electrical characteristics.

In an embodiment the esophageal carrying structure comprises a balloon and the electrodes are arranged on an outer surface of the balloon. The balloon is preferentially inflatable by filling a fluid like air or saline into the balloon, wherein, after the balloon has been inflated, the electrodes are in contact with the wall of the esophagus for measuring the electrical characteristics on the wall.

In another embodiment the esophageal carrying structure is linear or planar or partly cylindrical or fully cylindrical. Correspondingly, the electrodes may be arranged in a line or in a plane or partly cylindrically, especially half-cylindrically, or fully cylindrically. The partly or fully cylindrical structures are preferentially hollow. If the esophageal carrying structure is shaped in this way, in the esophagus an opening for saliva is left. This may allow performing the determination of the electrical characteristics without requiring an anesthesiologist.

The system may further comprise a proximity determination unit for determining whether an interventional instrument being adapted to electrically treat the heart is in proximity of the esophagus based on the electrical characteristics measured by the esophageal electrode structure within the esophagus. Since the interventional instrument is adapted to electrically treat the heart, the electrical instrument can influence the electrical characteristics measured by the esophageal electrode structure, wherein the proximity determination unit can determine that the interventional instrument is close to the esophagus, if the influence of the electrical characteristics measured by the esophageal electrode structure has been detected. If the proximity determination unit determines that the interventional instrument is close to the esophagus, this can be output to a physician using the interventional instrument for performing an interventional procedure, in order to avoid damaging of the esophagus. For instance, the interventional instrument may be an ablation catheter for ablating the heart, wherein, if the ablation catheter is close to the esophagus, the physician can be warned accordingly.

In a further preferred embodiment the esophageal electrode structure is adapted to cool the esophagus. For instance, the esophageal electrode structure can comprise electrodes and a balloon carrying the electrodes, wherein the balloon may be arranged to be filled with a cooling fluid. The cooling fluid may be gaseous or liquid. For instance, it may be air or saline. The cooling fluid may also be used for inflating the balloon. Cooling the esophagus is especially helpful, if an inner wall of the heart is heated for treating the same. In this case the cooling of the esophagus can strongly reduce the likelihood of damaging the esophagus by the heat.

In a further aspect of the present invention an interventional system for performing an interventional procedure, especially an electrophysiological (EP) procedure, within a heart of a living being is presented, wherein the interventional system comprises:

an interventional instrument being adapted to be introduced into the heart of the living being,

a system for determining electrical characteristics on a surface of the heart as defined in claim 1.

In another aspect of the present invention a method for determining electrical characteristics on a surface of a heart of a living being is presented, wherein the method comprises:

measuring electrical characteristics within the esophagus of the living being by using an esophageal electrode structure, which has been introduced into the esophagus,

determining the position of the esophageal electrode structure within the esophagus and the position of the surface of the heart by a position determination unit,

determining electrical characteristics on the surface of the heart based on the electrical characteristics measured by the esophageal electrode structure and based on the determined positions of the esophageal electrode structure and the surface of the heart by an electrical characteristics determination unit.

In a further aspect of the present invention a computer program for determining electrical characteristics on a surface of a heart of a living being is presented, wherein the computer program comprises program code means for causing a system as defined in claim 1 to carry out the steps of the method as defined in claim 14, when the computer program is run on a computer controlling the system.

It shall be understood that the system of claim 1, the interventional system of claim 13, the method of claim 14 and the computer program of claim 15 have similar and/or identical preferred embodiments, in particular, as defined in the dependent claims.

It shall be understood that a preferred embodiment of the invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows schematically and exemplarily an embodiment of an interventional system for performing an interventional procedure within a heart of a living being,

FIG. 2 schematically and exemplarily shows an esophageal electrode structure of the interventional system,

FIG. 3 schematically and exemplarily illustrates a preferred position of the esophageal electrode structure relative to the heart of the living being,

FIG. 4 shows a flowchart exemplarily illustrating an embodiment of a method for determining electrical characteristics on a surface of a heart of a living being,

FIGS. 5 to 12 schematically and exemplarily illustrate further embodiments of the esophageal electrode structure,

FIG. 13 shows schematically and exemplarily a further embodiment of an interventional system for performing an interventional procedure within a heart of a living being, and

FIGS. 14 and 15 schematically and exemplarily illustrate a further embodiment of the esophageal electrode structure.

### DETAILED DESCRIPTION OF EMBODIMENTS

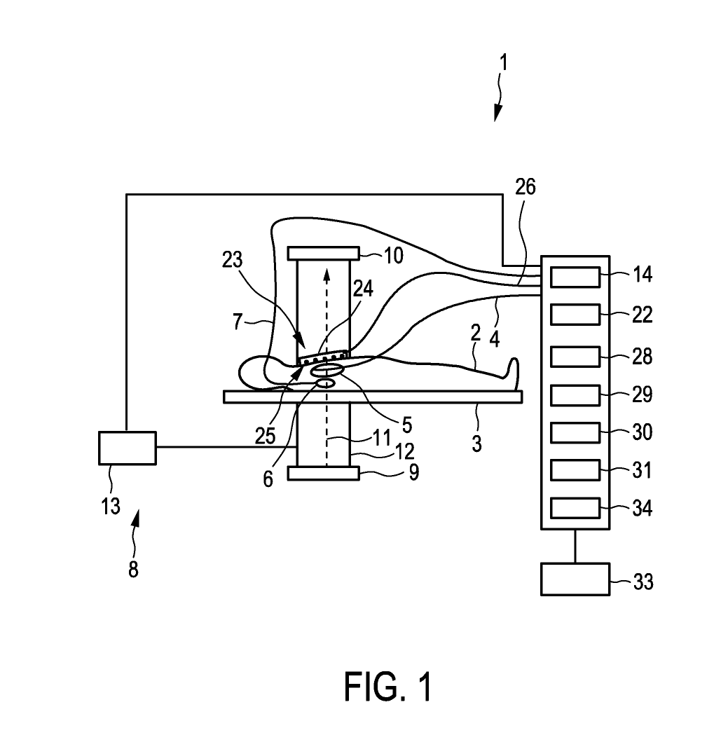

FIG. 1 shows schematically and exemplarily an embodiment of an interventional system for performing an interventional procedure within a heart of a person. In this embodiment the interventional system 1 is adapted to perform a cardiac ablation procedure within the heart 5 of the person 2 lying on a support means like a table 3. The ablation catheter 4 has been introduced into the heart 5 for ablating cardiac tissue. The catheter 4 is connected to a treating unit 31 being, in this embodiment, a radiofrequency (RF) source, in order to ablate the cardiac tissue by RF energy.

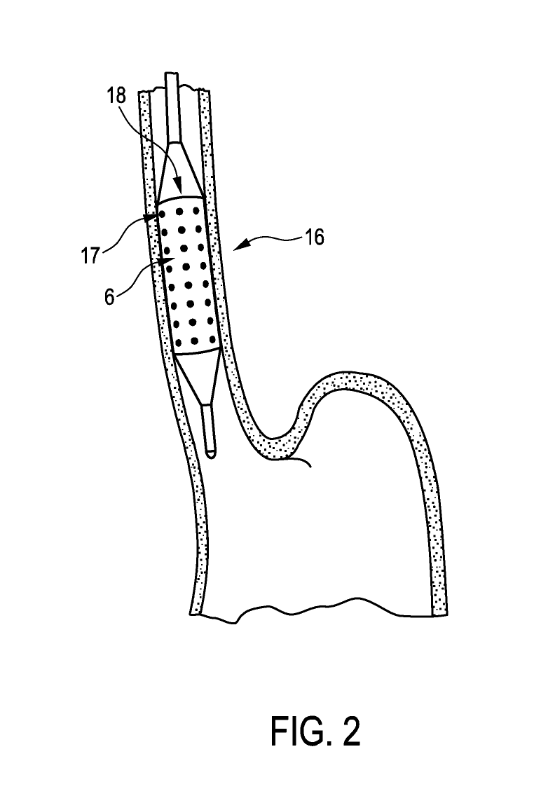

The interventional system 1 further comprises an esophageal electrode structure 6 introduced into the esophagus of the person 2 for measuring electrical potentials within the esophagus. The esophageal electrode structure 6 is exemplarily and schematically shown in more detail within the esophagus 16 in FIG. 2.

The esophageal electrode structure 6 comprises electrodes 17 and an esophageal carrying structure 18 carrying the electrodes 17. In this embodiment the esophageal carrying structure 18 is an inflatable balloon, wherein the electrodes 17 are arranged on an outer surface of the balloon. The balloon 18 is connected to a fluid providing unit 30 via a catheter 7 such that the balloon 18 is inflatable by filing the balloon with the fluid. The fluid providing unit 30 may comprise a fluid reservoir comprising the fluid and a pump for pumping the fluid into the balloon 18 for inflating the same. If the balloon 18 is inflated, the electrodes 17 are in contact with the wall of the esophagus 16.

A set of different esophageal electrode structures having different sizes may be provided. For instance, esophageal electrode structures may be provided, which have diameters ranging from 1 mm to 5 mm in the non-inflated state and diameters ranging from 10 to 30 mm in the inflated state. This allows the user to select the size of the esophageal electrode structure according to the anatomy of the person. Moreover, the set of esophageal electrode structures can be configured such that the distance between the electrodes is constant or varies with increasing diameter of the balloon of the respective esophageal electrode structure in the set of esophageal electrode structures.

The interventional system 1 further comprises a measuring unit 14 for measuring electrical potentials on the wall of the esophagus 16 by using the electrodes 17, when they are in contact with the wall of the esophagus 16. The electrodes 17 are connected with the measuring unit 14 via wires integrated in the catheter 7.

The interventional system 1 further comprises an outer electrode structure 23 arranged on the anterior surface of the thorax of the person 2. The outer electrode structure 23 comprises a carrying structure 24 carrying several electrodes 25 for measuring electrical potentials on the outer surface of the person 2. The outer electrode structure 23 may be regarded as being a patch comprising the electrodes. The electrodes 25 of the outer electrode structure 23 are connected to a measuring unit 22 via a cable 26 for measuring the electrical potentials on the outer surface of the person 2.

The interventional system further comprises an imaging unit 8 for generating image data showing the esophageal electrode structure 6 within the esophagus 16, the outer electrode structure 23 on the outer surface of the person 2 and the surface of the heart 5, on which electrical potentials should be determined. In this embodiment it is a surface of the left atrium of the heart 5, on which the electrical potentials should be determined.

The imaging unit 8 is a C-arm x-ray unit for generating a three-dimensional image showing the esophageal electrode structure 6 within the esophagus 16, the outer electrode structure 23 on the outer surface of the person 2 and the surface of the heart 5. The C-arm x-ray unit 8 comprises an x-ray source 9 for emitting x-rays 11 traversing the person 2 and an x-ray detector 10 for detecting the x-rays 11 after having traversed the person 2. The x-ray source 9 and the x-ray detector 10 are mounted on a C-arm 12. The C-arm 12 is rotatable around different axes, in order to acquire x-ray projection data in different acquisition directions. The acquired x-ray projection data are provided to an imaging control unit 13, which is adapted to control the C-arm x-ray unit 8 and to reconstruct a three-dimensional image based on the x-ray projection data acquired in the different acquisition directions. For reconstructing the three-dimensional image known reconstruction algorithms can be used like a filtered back projection algorithm, a Radon inversion algorithm, et cetera. The reconstruction may be performed ungated or using hyper pacing. Moreover, a contrast agent may have been injected and may be present in the left atrium, in order to allow the C-arm x-ray unit 8 to generate a three-dimensional image showing a contrast-enhanced surface of the left atrium. The C-arm x-ray unit 8 may especially be adapted to perform a rotational angiography acquisition, i.e. to reconstruct a three-dimensional image from a set of x-ray projections, especially for the atria, in order to perform 3D ATG.

The interventional system 1 further comprises a position determination unit 34 for determining the positions of the esophageal electrode structure 6, the outer electrode structure 23 and the surface of the heart 5 based on the three-dimensional image generated by the C-arm x-ray unit 8. In particular, the position determination unit 34 is adapted to segment the esophageal electrode structure 6, the outer electrode structure 23 and the surface of the heart 5 in the three-dimensional image for determining their positions. The determination of the position of the surface of the heart 5 includes the determination of the position of each part of the surface of heart, on which the electrical characteristics should be determined, wherein the positions of the parts of the surface of the heart define the location, orientation and shape of the surface. Moreover, the determined positions can be relative positions, for instance, the positions of the electrode structures can be positions relative to the position of the heart, or the determined positions can be absolute positions.

The interventional system 1 further comprises an electrical characteristics determination unit 28 for determining the electrical characteristics on the surface of the heart 5 based on the electrical characteristics measured by the esophageal electrode structure 6, the electrical characteristics measured by the outer electrode structure 23 and the determined positions. In this embodiment the electrical characteristics determination unit 28 is adapted to determine electrical potentials on the surface of the left atrium of the heart 5 based on electrical potentials measured by the esophageal electrode structure 6 and the outer electrode structure 23 and based on the determined positions. For determining the electrical characteristics on the surface of the heart based on electrical characteristics measured on the outer surface of the person 2 and on the wall of the esophagus 16 known algorithms can be used like the algorithm disclosed in the article “Electrocardiographic Imaging (ECGI): A Noninvasive Imaging Modality for Cardiac Electrophysiology and Arrhythmia” by Ramanathan et al., Nature Medicine 10, 22-428 (2004) or disclosed in U.S. Pat. No. 7,471,971, which are herewith incorporated by reference.

The electrical potentials determined for the surface of the left atrium can be shown together with, for instance, an image of the heart of the person 2 on a display 33.

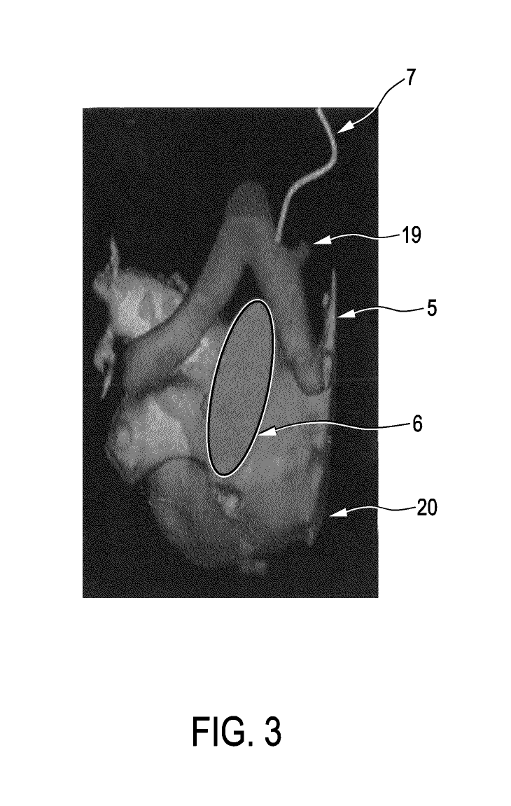

FIG. 3 schematically and exemplarily illustrates a preferred position of the esophageal electrode structure 6 relative to the heart 5, wherein reference number 19 indicates the bronchi and reference number 20 indicates the left atrium of the heart.

The interventional system 1 further comprises a proximity determination unit 29 for determining whether the ablation catheter 4 is close to the esophagus 16 of the person 2 based on the electrical characteristics, i.e. in this embodiment the electrical potentials, measured by the esophageal electrode structure 6 within the esophagus 16. For instance, the proximity determination unit 29 can be adapted to determine that the ablation catheter 4 is close to the esophagus 16, if the measured electrical potentials are larger than a predefined threshold. In this case a warning may be given to the physician via the display 33 or via another output unit providing a visual or acoustical output.

In this embodiment the fluid providing unit 30 is adapted to provide cooling fluid, which may be air or saline, for inflating the balloon 18. The esophageal electrode structure 6 can therefore be used to cool the esophagus 16 during the ablation procedure, thereby reducing the likelihood of damaging the esophagus 16 during the ablation procedure. Thus, in this embodiment the same fluid may be used for inflating the balloon 18 and for cooling the esophageal electrode structure 6 and thus the esophagus 16.

The esophageal electrode structure 6, the imaging unit 8, the position determination unit 34, the electrical characteristics determination unit 28 and the measuring units 14, 22 can be regarded as being components of a system for determining electrical characteristics on a surface of a heart of a living being, which in the above described embodiment is integrated with the interventional system 1. In another embodiment the system for determining electrical characteristics on a surface of a heart of a living being can be a separate system, i.e. it may not be integrated with an interventional system.

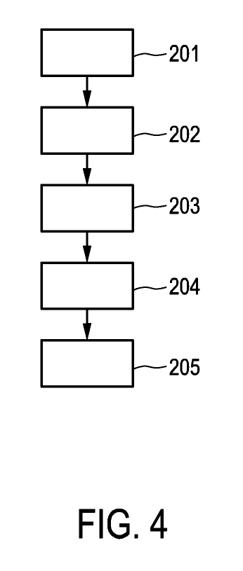

In the following an embodiment of a method for determining electrical characteristics on a surface of a heart of a living being will exemplarily be described with reference to a flowchart shown in FIG. 4.

In step 201 electrical characteristics within the esophagus 16 of the person 2 are measured by using the esophageal electrode structure 6 and electrical characteristics on the outer surface of the person 2 are measured by the outer electrode structure 23. In step 202 the imaging unit 8 generates image data, which show the esophageal electrode structure 6 in the esophagus 16, the outer electrode structure 23 on the outer surface of the person 2 and the surface of the heart 5 on which the electrical characteristics should be determined. In step 203 the position determination unit 34 determines the positions of the esophageal electrode structure 6, the outer electrode structure 23 and the surface of the heart 5 based on the generated image data. For instance, the esophageal electrode structure 6, the outer electrode structure 23 and the surface of the heart 5 can be segmented in the generated image data for determining their positions. In step 204 the electrical characteristics on the surface of the heart 5 are determined based on these positions and the electrical characteristics measured by the esophageal electrode structure 6 and the outer electrode structure 23 by the electrical characteristics determination unit 28. In step 205 the determined electrical characteristics are shown on the display 33, for instance, as a colored map, wherein the electrical characteristics are color coded and shown on a three-dimensional image or model of the heart 5.

The steps of the method for determining the electrical characteristics on the surface of the heart 5 can also be performed in another order. For instance, steps 202 and 203 can be performed before step 201, or step 201 can be performed simultaneously with steps 202 and 203. Moreover, steps 201 to 205 can be performed in a loop such that at several times, in particular, continuously, the electrical characteristics are measured, the image data are generated, the positions are determined, and the electrical characteristics are determined and displayed. For instance, during an interventional procedure like an ablation procedure the display can continuously be updated based on the actually measured electrical characteristics and/or the actually generated image data, in order to allow the physician to perform the interventional procedure depending on the displayed actual electrical characteristics on the surface of the heart. The determined electrical characteristics on the surface of the heart can also be provided to a robot, which may perform the interventional procedure, in order to allow the robot to automatically perform the interventional procedure based on the determined electrical characteristics.

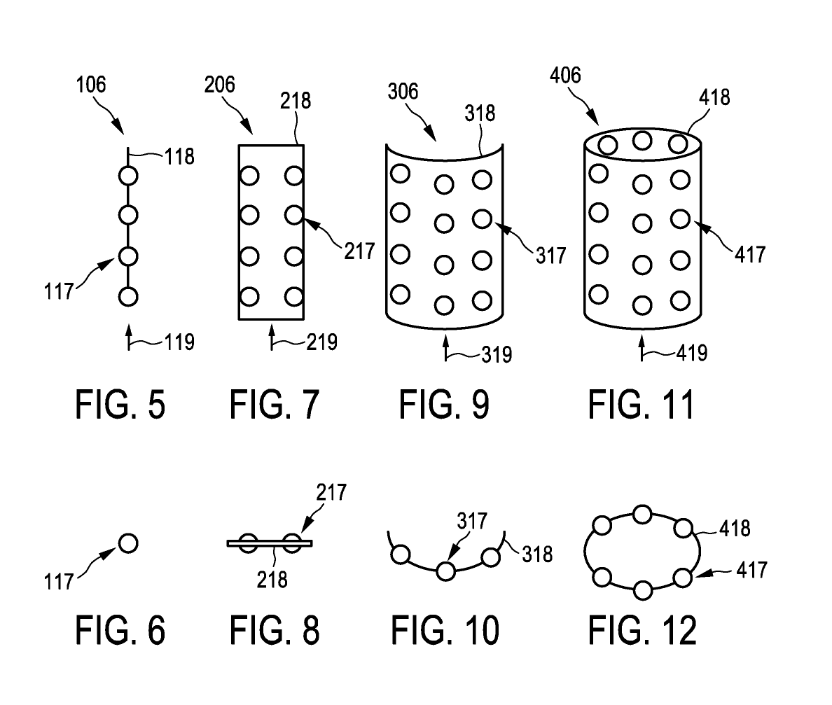

Although in above described embodiments the esophageal electrode structure has a certain configuration, in other embodiments the esophageal electrode structure can have another configuration. For instance, the esophageal electrode structure can be configured as schematically and exemplarily shown in FIGS. 5 to 12. In particular, the esophageal carrying structure of the esophageal electrode structure may be linear, planar, partly cylindrical, especially half-cylindrical or fully cylindrical. Correspondingly, the electrodes may be arranged in a line, in a plane, partly cylindrically, especially half-cylindrically, or fully cylindrically. If the esophageal carrying structure is shaped in this way, an opening for saliva is left. This may allow for a determination of the electrical characteristics on the surface of the heart without requiring an anesthesiologist.

FIGS. 5 and 6 schematically and exemplarily illustrate a linear configuration of an esophageal electrode structure 106, wherein FIG. 5 is a side view and FIG. 6 is a view in the direction indicated in FIG. 5 by the arrow 119. The linear esophageal electrode structure 106 comprises electrodes 117 and a linear esophageal carrying structure 118. FIGS. 7 and 8 schematically and exemplarily illustrate a planar esophageal electrode structure 206 comprising a planar esophageal carrying structure 218 and electrodes 217, wherein FIG. 7 is a side view and FIG. 8 is a view in the direction indicated in FIG. 7 by the arrow 219. FIGS. 9 and 10 schematically and exemplarily illustrate a semi-cylindrical esophageal electrode structure 306 comprising a semi-cylindrical esophageal carrying structure 318 and electrodes 317, wherein FIG. 9 is a side view and FIG. 10 is a view in the direction indicated in FIG. 9 by the arrow 319. FIGS. 11 and 12 schematically and exemplarily illustrate a cylindrical esophageal electrode structure 406 comprising a cylindrical esophageal carrying structure 418 and electrodes 417, wherein FIG. 11 is a side view and FIG. 12 is a view in the direction indicated in FIG. 11 by the arrow 419. FIGS. 5 to 12 just illustrate preferred shapes of the esophageal electrode structure, wherein further components like electrical wires for connecting the electrodes are not shown in these figures for clarity reasons.

The esophageal electrode structures schematically and exemplarily illustrated in FIGS. 5 to 12 do not fill the complete esophagus, i.e., for instance, the semi-cylindrical esophageal carrying structure shown in FIGS. 9 and 10 and the cylindrical esophageal electrode structure shown in FIGS. 11 and 12 are hollow semi-cylindrical and hollow cylindrical structures, respectively. Saliva can therefore pass the esophageal electrode structure such that an anesthesiologist may not be required during electrocardiographic imaging.

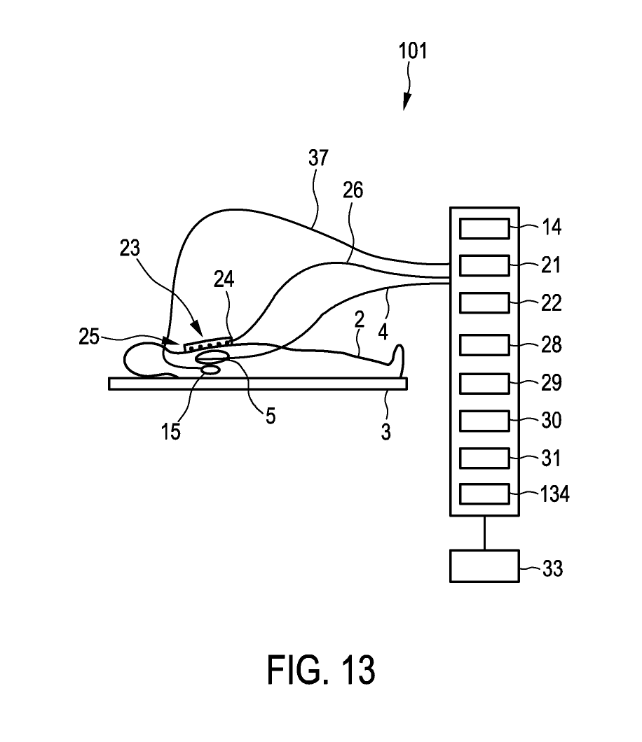

FIG. 13 shows schematically and exemplarily a further embodiment of an interventional system for performing an interventional procedure within a heart of a living being. The system 101 schematically and exemplarily shown in FIG. 4 is similar to the interventional system 1 described above with reference to FIGS. 1 to 3, except for the imaging unit, i.e. in this embodiment 101 the imaging unit is not a C-arm x-ray unit, but a TEE ultrasound imaging unit. The TEE ultrasound imaging unit comprises a TEE ultrasound probe, which is integrated with the esophageal electrode structure for forming a combined TEE ultrasound probe and esophageal electrode structure device 15. The combined device 15 is introduced into the esophagus and connected via an electrical connection within a catheter 37 to an ultrasound image generation unit 21. The TEE ultrasound probe, which might be a micro TEE ultrasound probe or another TEE ultrasound probe, generates TEE ultrasound signals, which are used by the ultrasound image generation unit 21 for generating a three-dimensional ultrasound image showing cardiac tissue and the surface of the heart 5.

In this embodiment the spatial relation between the TEE ultrasound probe and the esophageal electrode structure on the combined device is known, wherein a position determination unit 134 is adapted to determine the position of the esophageal electrode structure relative to the image data generated by the TEE ultrasound probe based on the known spatial relation between the TEE ultrasound probe and the esophageal electrode structure. Moreover, the position determination unit 134 is adapted to determine the position of the surface of the heart 5 by segmenting the surface in the ultrasound image data set. Furthermore, the position determination unit 134 is adapted to determine the position of the outer electrode structure 23 relative to the position of the combined device 15 by optical shape sensing, wherein the catheter 37 and the cable 26 are equipped with optical shape sensing fibers for generating optical shape sensing signals used by the position determination unit 134 for determining the absolute positions of the combined device 15 and the outer electrode structure 23 and wherein these absolute positions can be used for determining the relative position between the combined device 15 and the outer electrode structure 23. In another embodiment the ultrasound image generated by the TEE ultrasound probe may also show the outer electrode structure 23, wherein in this case the position of the outer electrode structure 23 can be determined by segmenting the outer electrode structure 23 in the ultrasound image. An additional tracking technique like the above described optical shape sensing technique may then not be needed.

Based on the positions and the electrical potentials measured by the esophageal electrode structure 6 and the outer electrode structure 23 the electrical potentials on the surface of the heart 5 are determined by the electrical characteristics determination unit 28.

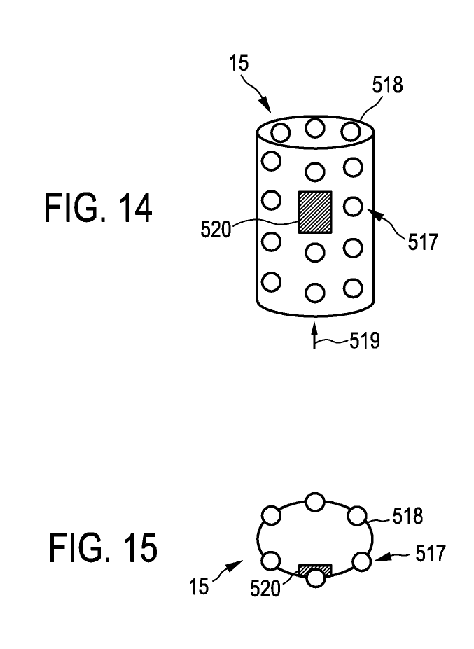

FIGS. 14 and 15 schematically and exemplarily illustrate an embodiment of the combined device 15, wherein FIG. 14 shows a side view and FIG. 15 shows a view in the direction indicated in FIG. 14 by the arrow 519. The combined device 15 comprises a carrying structure 518, which is cylindrical in this embodiment, wherein the carrying structure 518 carries electrodes 517 and the TEE ultrasound probe 520. FIGS. 14 and 15 just illustrate exemplarily a certain structure of the combined device comprising the esophageal electrode structure and the TEE ultrasound probe, wherein further components like electrical wires for connecting the electrodes and for connecting the TEE ultrasound probe are not shown in these figures for clarity reasons. In another embodiment the combined device can be constructed in another way. For instance, the electrodes may be provided on the surface of the TEE ultrasound probe, in order to provide a combined esophageal electrode structure and TEE ultrasound probe device. In particular, the TEE ultrasound probe may comprise a matrix of ultrasound transducers, wherein electrodes may be arranged around the matrix and possibly at further locations on the TEE ultrasound probe. In a further embodiment of the combined device the esophageal electrode structure may be arranged adjacent to the TEE ultrasound probe. For instance, an inflatable balloon with outer electrodes forming the esophageal electrode structure can be arranged adjacent to the TEE ultrasound probe.

Known electrocardiographic imaging systems measure body surface potentials with an electrode array covering the human thorax. For instance, these systems may comprise a vest with 150 to 250 electrodes. By using the measured potential data in combination with person-specific anatomical data of the person's thorax, which may be obtained from a computed tomography image, the electrical activity of the heart can be reconstructed noninvasively. This determination of the electrical activity may be performed for diagnostic purposes and/or during interventional procedures like EP interventions, in order to guide the treatment and to evaluate the treatment success. However, since the accuracy of the electrocardiographic reconstruction is inversely proportionally related to the squared distance of the measuring electrodes to the surface of the heart on which, for instance, a current distribution should be reconstructed, wherein this distance is relatively large, and since the electrocardiographic reconstruction depends on the tissue types and tissue conductivities between the heart surface and the electrodes, which are generally not exactly known, the accuracy of determining the electrical characteristics on the surface of the heart is not very high. Moreover, if a C-arm x-ray unit with a rotational angiography acquisition is used for reconstructing an anatomical road map for ablation guidance during an ablation procedure, only a limited field of view can be reconstructed based on the acquired x-ray data. This limited reconstructed field of view may contain only the left atrium of the heart and some surrounding anatomy and some of the set of electrodes on the thorax, for instance, all 150 to 250 electrodes of the vest, may not be visible in the reconstructed field of view. Thus, if the C-arm x-ray unit with the rotational angiography acquisition used for the ablation procedure, in order to reconstruct the anatomical road map for ablation guidance, should also be used for determining the electrical characteristics on the surface of the heart, only few electrodes of the electrodes arranged on the thorax surface may be considered, thereby further decreasing the accuracy of determining the electrical characteristics on the surface of the heart. Alternatively, it may be required to use an additional imaging unit for imaging all electrodes on the thorax surface, but using two different imaging units is technically more complex and cumbersome for the physician.

The systems for determining the electrical characteristics on the surface of the heart described above with reference to FIGS. 1 and 13 are therefore configured such that a subset of all electrodes used for the electrocardiographic imaging is arranged on an esophageal catheter, in particular, on a carrying structure of the esophageal catheter forming together with the subset of the electrodes the esophageal electrode structure, in order to position the subset of electrodes in the esophagus close to, for instance, the left atrium. Since for this subset of electrodes the distance to the surface of the heart has been reduced and since less tissue with different conductivities is present between this subset of electrodes and the target reconstruction surface, i.e. the surface of the heart on which the electrical characteristics should be determined, the accuracy of determining the electrical characteristics on the surface of the heart can be improved. Moreover, the subset of electrodes can be positioned within the esophagus such that it is within the limited reconstructed field of view that is available, if a C-arm x-ray unit is used with a rotational angiography acquisition for generating the image data. The remaining electrodes on the thorax surface of the person can also be arranged such that they are within this limited reconstructed field of view, i.e. all electrodes may be arranged within the limited reconstructed field of view such that the electrical characteristics on the surface of the heart can very accurately be determined during an interventional procedure like an ablation procedure already using the C-arm x-ray unit with the rotational angiography acquisition, without requiring an additional imaging unit. For instance, a set of electrodes can be arranged on a balloon which can be positioned within the esophagus, and a small patch of further electrodes can be arranged on the anterior surface of the thorax such that all electrodes are still inside the limited reconstruction volume. This allows localizing all electrodes in their relation to the surface of the heart, in particular, to the surface of the left atrium, using the image data.

The systems described above with reference to FIGS. 1 and 13 are especially suited for performing electrocardiographic imaging of the left atrium during EP interventions, in order to monitor, for instance, therapy effects, wherein combined measurements from two sets of electrodes are used. They comprise an esophageal balloon catheter which can be introduced through the mouth or the nose into the esophagus. The esophageal balloon catheter preferentially comprises a small diameter catheter with an inflatable balloon and electrodes on the surface of the balloon. The diameter of the balloon in the non-inflated state may be equal to or smaller than 5 mm, whereas in its inflated state the balloon may have a diameter within the range of 10 to 30 mm. The balloon is preferentially configured such that, if the balloon is inflated, the electrodes tightly connect to the surface of the esophagus, wherein during this tight contact situation potential measurements may be performed.

In the embodiments described above with reference to FIGS. 1 and 13 the balloon is combined with a patch of electrodes, i.e. the outer electrode structure, on the person's front side, in order to integrate measurements from both sides of the left atrium into the electrocardiographic imaging. Preferentially the electrocardiographic imaging is performed during an EP intervention, wherein the patch is attached to the surface of the person and the balloon is positioned in the esophagus. In the embodiment described above with reference to FIG. 1 a rotational angiography acquisition of the left atrium may be performed (3D ATG) for generating image data, in which the surface of the left atrium may be segmented and the position of the electrodes on the balloon and the patch may be localized. Using the potential measurements of the electrodes and knowing their position in relation to the left atrium, the current distribution or another electrical characteristic on the surface of the left atrium can be reconstructed throughout the EP intervention, in particular, throughout an ablation intervention. The balloon can also be used to determine the proximity of the ablation catheter to the esophagus, in order to avoid damaging of the esophagus by, for instance, RF ablation. For determining the proximity the above described proximity determination unit 29 may be used. However, also without explicitly determining the proximity of the ablation catheter, a warning may be given to the physician, if the ablation catheter is close to the esophagus. For instance, if during the ablation procedure the electrical characteristics of the surface of the left atrium are continuously determined and shown on the display, this continuous determination of the electrical characteristics may be strongly disturbed, if the ablation catheter is close to the esophageal electrode structure within the esophagus and if stimulation pulses are applied to the ablation catheter. This strong disturbance may be visible on the display 33 showing the determined electrical characteristics on the surface of the left atrium, i.e. on the generated electrocardiographic image. In particular, stimulation pulses applied to the ablation catheter may light up in the electrocardiographic image. If ablation close to the esophagus is required, the balloon may also be used to cool the ablation site to avoid damaging of the esophagus.

In an embodiment the system for determining the electrical characteristics on the surface of the heart may further comprise a tracking unit for tracking movements of the esophageal electrode structure and/or of the outer electrode structure, which may be caused by breathing or by cardiac motion. The tracked motion of the esophageal electrode structure and/or of the outer electrode structure may be used for correcting the positions of these electrode structures relative to the surface of the heart, on which the electrical characteristics should be determined, especially during an interventional procedure. The tracking unit can be adapted to track the motion of the electrode structures by using an optical shape sensing tracking technique, an electromagnetic tracking technique or any other tracking technique. In the embodiment described above with reference to FIG. 13 the tracking unit may be the position determination unit that determines the positions of the esophageal electrode structure and the outer electrode structure.

In an embodiment only the motion of the outer electrode structure is tracked and this tracked motion is used for correcting the position of the outer electrode structure relative to the surface of the heart. The tracking only of the outer electrode structure can still result in good motion correction, if it can be assumed that the outer electrode structure is moved due to respiratory motion only and the esophageal electrode structure and the surface of the heart have a fixed spatial relation.

Although in above described embodiments the electrical characteristics on the surface of the heart have been determined during an ablation procedure, in other embodiments the electrical characteristics on the surface of the heart can be determined during another interventional procedure like a cardiac resynchronization therapy (CRT) procedure or a ventricular tachycardia (VT) treatment procedure, wherein in these cases preferentially the complete heart is imaged and the resulting image data are used for determining the positions of the electrode structures and the surface of the heart, on which the electrical characteristics should be determined. The electrical characteristics on the surface of the heart can also be determined without simultaneously performing an interventional procedure. For instance, the electrical characteristics on the surface of the heart can be determined for diagnostic purposes, wherein in this case it may not be required to insert any instrument into the heart.

In a further embodiment further electrodes may be placed at further positions close to the heart, wherein also these additional electrodes may be used for measuring electrical characteristics, wherein also the positions of these further electrodes may be determined and wherein the electrical characteristics measured by these further electrodes and their positions may additionally be used for determining the electrical characteristics on the surface of the heart. For instance, catheters on a coronary sinus (CS) catheter may be used as further electrodes for performing the electrocardiographic imaging.

Although in an above described embodiment the interventional system is adapted to ablate cardiac tissue by using RF energy, in other embodiments the interventional system can be adapted to ablate cardiac tissue in another way. For instance, the interventional system can be adapted to use ablation techniques like cryo ablation, laser ablation, et cetera, wherein also while applying these ablation techniques the electrical characteristics on the surface of the heart may be monitored by using the esophageal electrode structure, the outer electrode structure and the positions of the esophageal electrode structure, the outer electrode structure and the surface of the heart.

Although in an above described embodiment a three-dimensional image has been reconstructed based on x-ray projection data generated by a C-arm x-ray unit by using a registration algorithm, wherein the positions of the electrodes and the surface of the heart have been determined by segmenting these components in the reconstructed three-dimensional image, in other embodiments the x-ray projection data can be used in another way for determining the positions of the electrodes and/or of the surface of the heart. For instance, the positions of the electrodes and/or of the surface of the heart may be detected in the x-ray projection data, which have been acquired in different acquisition directions, wherein these determined positions in the projection data and the corresponding acquisition geometry can be used for determining the three-dimensional positions of the electrodes and/or of the surface of the heart by using the epipolar geometry. This may allow for a determination of the three-dimensional positions of the electrodes and/or of the surface of the heart based on less x-ray projection data in comparison to x-ray projection data that need to be acquired, in order to reconstruct a three-dimensional image. Thus, the radiation dose applied to the person may be reduced.

In a further embodiment the position determination unit may be adapted to determine the position of the surface of the heart based on tracked positions of a catheter like an ablation catheter, wherein these tracked positions of the catheter have been determined, while the catheter is in contact with the surface of the heart. For instance, the catheter may be moved along the surface of the heart, while the position of the catheter is tracked, in order to provide tracked positions of the catheter, which can be used by the position determination unit for determining the position of the surface of the heart.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Procedures like reconstructing an image, determining positions, determining electrical characteristics, et cetera, performed by one or several units or devices can be performed by any other number of units or devices. These procedures and/or the control of the system for determining electrical characteristics on a surface of a heart of a living being in accordance with the method for determining electrical characteristics on a surface of a heart of a living being and/or the control of the interventional system can be implemented as program code means of a computer program and/or as dedicated hardware.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

Any reference signs in the claims should not be construed as limiting the scope.

The invention relates to a system for determining electrical characteristics like electrical potentials on a surface of a heart. An esophageal electrode structure measures electrical characteristics within an esophagus and a position determination unit determines the position of the esophageal electrode structure within the esophagus and the position of the surface of the heart. The electrical characteristics on the surface of the heart are then determined based on the measured electrical characteristics and based on the determined positions of the esophageal electrode structure and the surface of the heart. Since for measuring the electrical characteristics the esophageal electrode structure is used, the electrical characteristics can be measured within the esophagus and thus close to the surface of the heart, thereby allowing for an improved accuracy of determining the electrical characteristics on the surface of the heart.

1. A system for determining electrical characteristics on a surface of a heart of a living being, wherein the system comprises:
an esophageal electrode structure being adapted to be introduced into the esophagus of the living being and to measure electrical characteristics within the esophagus,
a position determination unit for determining the position of the esophageal electrode structure and the position of the surface of the heart,
an electrical characteristics determination unit for determining electrical characteristics on the surface of the heart based on the electrical characteristics measured by the esophageal electrode structure and based on the determined positions of the esophageal electrode structure.

2. The system as defined in claim 1, wherein the system further comprises an outer electrode structure being adapted to be arranged on an outer surface of the living being, wherein the position determination unit is adapted to determine also the position of the outer electrode structure on the outer surface of the living being and wherein the electrical characteristics determination unit is adapted to determine the electrical characteristics on the surface of the heart based on the electrical characteristics measured by the esophageal electrode structure, the electrical characteristics measured by the outer electrode structure and the determined positions.

3. The system as defined in claim 2, wherein the system further comprises an imaging unit for generating image data showing the outer electrode structure on the outer surface of the living being, wherein the position determination unit is adapted to determine the position of the outer electrode structure on the outer surface of the living being based on the generated image data.

4. The system as defined in claim 1, wherein the system further comprises an imaging unit for generating image data showing the esophageal electrode structure and/or the surface of the heart, wherein the position determination unit is adapted to determine the position of the esophageal electrode structure and/or of the surface of the heart based on the generated image data.

5. The system as defined in claim 4, wherein the imaging unit is a C-arm x-ray unit for generating a three-dimensional image as the image data.

6. The system as defined in claim 4, wherein the imaging unit comprises a transesophageal echocardiogram ultrasound probe adapted to be introduced into the esophagus of the living being and to generate an ultrasound image as the image data.

7. The system as defined in claim 6, wherein the transesophageal echocardiogram ultrasound probe and the esophageal electrode structure are integrated with each other.

8. The system as defined in claim 1, wherein the esophageal electrode structure comprises electrodes and an esophageal carrying structure being adapted to carry the electrodes and to be introduced into the esophagus of the living being.

9. The system as defined in claim 8, wherein the esophageal carrying structure comprises a balloon and wherein the electrodes are arranged on an outer surface of the balloon.

10. The system as defined in claim 8, wherein the esophageal carrying structure is linear or planar or partly cylindrical or fully cylindrical.

11. The system as defined in claim 1, wherein the position determination unit is adapted to determine the position of the esophageal electrode structure by optical shape sensing or by electromagnetic tracking.

12. The system as defined in claim 1, wherein the esophageal electrode structure is adapted to cool the esophagus.

13. An interventional system for performing an interventional procedure within a heart of a living being, the interventional system comprising:
an interventional instrument being adapted to be introduced into the heart of the living being,
a system for determining electrical characteristics on a surface of the heart as defined in claim 1.

14. A method for determining electrical characteristics on a surface of a heart of a living being, wherein the method comprises:
measuring electrical characteristics within the esophagus of the living being by using an esophageal electrode structure, which has been introduced into the esophagus,
determining the position of the esophageal electrode structure within the esophagus and the position of the surface of the heart by a position determination unit,
determining electrical characteristics on the surface of the heart based on the electrical characteristics measured by the esophageal electrode structure and based on the determined positions of the esophageal electrode structure and the surface of the heart by an electrical characteristics determination unit.

15. A computer program for determining electrical characteristics on a surface of a heart of a living being, the computer program comprising program code means for causing a system to carry out the steps of the method as defined in claim 14, when the computer program is run on a computer controlling the system.
